SEQUENCE LISTING (4 sequences)

SEQ ID NO: 1 - DNA, 31 bp, Artificial Sequence
  PCR primer
gcgaattcatgagtaagatttttgaagataa

SEQ ID NO: 2 - DNA, 32 bp, Artificial Sequence
  PCR primer
gcgaattctatatactgttgcaattctttctc

SEQ ID NO: 3 - DNA, 35 bp, Artificial Sequence
  PCR primer
ggctagcattaatgatatgggaactgaagaaagat

SEQ ID NO: 4 - DNA, 30 bp, Artificial Sequence
  PCR primer
gccggatccttattaactgcagggcacaga

(19) United States
(12) Patent Application Publication
(10) Pub. No.: US 2004/0157290 A1
(43) Pub. Date: Aug. 12, 2004

(54) Process for preparing serine-rich protein employing cysteine synthase (cysK) gene

(75) Inventors: Sang Yup Lee, Yuseong-gu (KR); Mee-Jung Han, Yuseong-gu (KR)

Correspondence Address: SUGHRUE MION, PLLC, 2100 PENNSYLVANIA AVENUE, N.W., SUITE 800, WASHINGTON, DC, 20037, US

(73) Assignee: KOREA ADVANCED INSTITUTE OF SCIENCE AND TECHNOLOGY

(21) Appl. No.: 10/662,517

(22) Filed: Sep. 16, 2003

(30) Foreign Application Priority Data
Feb. 12, 2003 (KR) 10-2003-0008689

(51) Int. Cl. C07K 14/52; C07H 21/04; C12N 1/21

(52) U.S. Cl. 435/69.1; 435/320.1; 435/252.33; 530/351; 536/23.5

(57) ABSTRACT

The present invention relates to a process for preparing a foreign protein comprising culturing a bacterium containing the cysteine synthase (cysK) gene and a gene encoding the foreign protein. The present invention comprises the steps of culturing a bacterium transformed with an expression vector containing a gene encoding a serine-rich foreign protein and an expression vector containing the cysK gene, or a bacterium transformed with an expression vector containing the cysK gene and a gene encoding a serine-rich foreign protein and isolating the foreign protein therefrom. The present invention is expected to be widely used to increase the production yield of a serine-rich foreign protein.

The present invention relates to a process for preparing a serine-rich protein comprising culturing a bacterium containing the cysteine synthase (cysK) gene and a gene encoding a foreign protein. More particularly, it relates to a process for preparing a serine-rich protein comprising culturing a bacterium containing a gene of a serine-rich foreign protein and the cysteine synthase (cysK) gene and isolating the serine-rich foreign protein therefrom.

### BACKGROUND ART

E. coli is a strain commonly used for synthesis and production of a foreign protein and has been applied in production of proteins, such as interferon, interleukin 2, colony-stimulating factors, growth hormones, insulin-like growth factors and human serum albumin, by recombinant technology. Also, for efficient production of a foreign protein in E. coli, plasmid vector expressing a foreign protein, proper culture conditions, inhibiting conditions of degradation of the prepared foreign, and the like are required and various systems have been developed to satisfy these requirements (Weickert et al., Curr. Opin. Biotechnol., 7:494-9, 1996).

However, there has been a problem in that when a method commonly used at present is used, it is hard to improve production yield of a foreign protein, because much time is required after induction of expression. Therefore, efforts have been made to overcome the problem but there has not been a report of a satisfactory result.

Therefore, there is a continuous need to develop a process for preparing a foreign protein by E. coli in a high yield.

Accordingly, the present inventors have conducted researches and studies to develop a method of producing a foreign protein by E. coli in a high yield. As a result, we have discovered that when the serine-rich foreign protein is prepared in E. coli, the production yield of a serine-rich foreign protein can be increased by coexpression of a gene encoding the serine-rich foreign protein and the cysteine synthase (cysK) gene derived from a bacterium and completed the present invention.

### SUMMARY OF THE INVENTION

Thus, it is an object of the present invention to provide a process for preparing a foreign protein comprising culturing a bacterium containing the cysK gene and a gene encoding the foreign protein.

It is a further object of the present invention to provide a bacterium simultaneously transformed with a recombinant vector including a gene encoding a foreign protein and a recombinant vector including the cysK gene, and a bacterium transformed with a recombinant vector including both the cysK gene and a gene encoding a foreign protein.

It is another object of the present invention to provide a process for preparing a foreign protein using a microorganism transformed with the cysK gene or a recombinant vector including the cysK gene.

In accordance with the present invention, the above and other objects can be accomplished by the provision of a process for preparing a foreign protein comprising culturing a bacterium containing the cysK gene and a gene encoding the foreign protein.

According to the present invention, the bacterium may be transformed with a vector containing both the cysK gene and a gene encoding the foreign protein. Alternatively, the bacterium may be transformed with a vector containing the cysK gene and a vector containing a gene encoding the foreign protein.

Also, in accordance with another aspect of the present invention, there is provided a recombinant vector containing the cysK gene and a gene encoding a foreign protein. In accordance with yet another aspect of the present invention, there is provided a bacterium transformed with a vector containing the cysK gene and a vector containing a gene encoding a foreign protein.

In accordance with yet another aspect of the present invention, there is provided a use of the cysK gene or a recombinant vector including the cysK gene in a method for preparing a foreign protein using a transformed microorganism.

According to the present invention, the cysK gene is derived from E. coli and the foreign protein is one selected from serine-rich proteins.

Also, the serine-rich proteins include leptin, IL-12p40 (interleukin 12 β chain), but are not limited thereto.

### BRIEF DESCRIPTION OF DRAWINGS

Further objects and advantages of the invention can be more fully understood from the following detailed description taken in conjunction with the accompanying drawings in which:

FIG. 1 is a graph showing expression levels of the GlyA and CysK proteins in cells before and after inducing expression of leptin protein using recombinant E. coli;

FIG. 2 is the gene map of the pAC104CysK plasmid;

FIG. 3 is the gene map of the pEDIL-12p40 plasmid;

FIG. 4a is a graph showing changes in cell density, dry cell weight and quantity of the foreign protein according to the culture time, when the recombinant E. coli BL21(DE3)(pEDOb5) that can produce leptin is cultured;

FIG. 4b is a graph showing changes in cell density, dry cell weight and quantity of foreign proteins according to the culture time, when the recombinant E. coli BL21(DE3)(pEDOb5)(pAC104CysK) that can produce leptin and coexpress the cysK gene is cultured;

FIG. 5a is a graph showing the amino acid composition of the E. coli proteins;

FIG. 5b is a graph showing the amino acid composition of leptin;

FIG. 5c is a graph showing the amino acid composition of the G-CSF;

FIG. 5d is a graph showing the amino acid composition of IL-12p40;

FIG. 6a is a graph showing changes in cell density, dry cell weight and quantity of foreign proteins according to the culture time, when the recombinant E. coli BL2I(DE3)(pEDIL-12p40) that can produce IL-12p40 is cultured; and

FIG. 6b is a graph showing changes in cell density, dry cell weight and quantity of foreign proteins according to the culture time, when the recombinant E. coli BL21(DE3)(pEDIL-12p40)(pAC104CysK) that can produce IL-12p40 and coexpress the cysK gene is cultured.

### DETAILED DESCRIPTION OF THE INVENTION

Now, the present invention will be described in detail.

Firstly, the terms used herein are defined as follows.

The term “specific amino acid rich protein” used herein refers to a protein containing a specific amino acid more than an average amino acid composition in E. coli (Koonin et al., in Escherichia coli and Salmonella: Cellular and Molecular Biology(eds. Neidhardt, F. C. et al.) American Society for Microbiology, Washington, D.C., 2203-17, 1996). The term “serine-rich protein” used here can be defined as a protein in which serine is contained in an amount of 10% or more of the amino acid composition and is ranked at least the second based on the content in the protein.

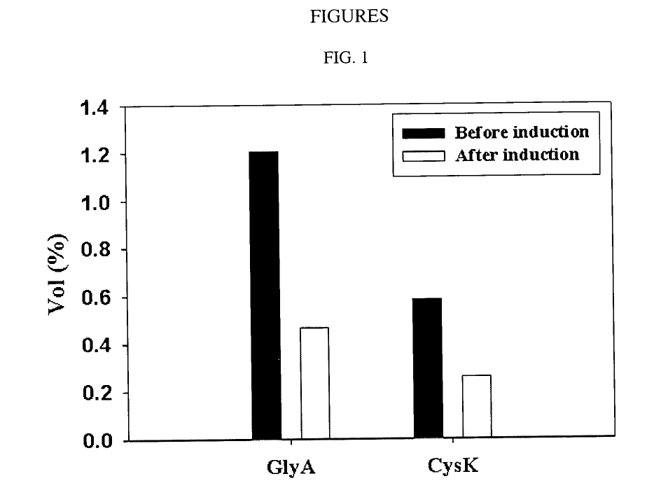

According to the fact known up to now, if a serine-rich protein such as leptin is produced in E. coli by DNA recombinant technology, the process for preparing leptin involves high concentration culturing of transformed E. coli, induction of leptin expression, expression of leptin, separation of transformed E. coli and extract of leptin from the E. coli. Here, over 8 hours are required for the production of leptin from the induction of expression to reach the maximum content. The present inventors have confirmed by two-dimensional electrophoresis that such a long time is required for the expression because biosynthetic pathway of serine family amino acids in E. coli is inhibited when leptin producing E. coli is cultured in a high concentration (FIG. 1).

Thus, the present inventors have discovered that it is possible to reduce the production time of a serine-rich protein by coexpressing the gene encoding cysteine synthase that promotes synthesis of serine family amino acids along with a gene of a serine-rich protein, as compared expressing only a gene of a serine-rich protein, thereby causing an increase in the production yield.

Now, the present invention will be described in detail by the following examples. However, it is apparent to those skilled in the art that the examples are only for illustrative purpose and the present invention is not limited thereto.

### EXAMPLE 1

Assay of Physiological Change of Leptin Protein Producing Strain Using Two-Dimensional Electrophoresis

The protein level changes before and after overproducing the human-derived leptin of E. coli BL21 (DE3)(pEDOb5) was compared by two-dimensional electrophoresis according to a known method (Hochstrasser et al., Anal. Biochem., 173:424-35, 1988; Han et al., J. Bacteriol., 183:301-8, 2001): That is, after pre-culturing of the E. coli BL21(DE3)(pEDOb5), expression of leptin was induced and cultured at a high concentration. A culture broth is taken before and after the induction of expression. Each culture broth is centrifuged at 6000 rpm for 5 minutes at 4° C. to obtain precipitations, which were washed with 500 μl of low salt buffer (KCl 3 mM, KH₂PO₄ 1.5 mM, NaCl 68 mM, NaH₂PO₄ 9 mM). Then, the product was suspended in 200 μl of TE buffer (Tris—HCl 10 mM, EDTA 1 mM). The suspension is sonicated with a sonicator and centrifuged at 12,000 rpm for 10 minutes at 4° C. The supernatant was discarded and the solid was dried in vacuo and stored at −20° C., which was used as a sample for the subsequent test.

200 μg of the prepared sample was dissolved in 340 μl of modified IEF solution (urea 9M, CHAPS 0.5%(w/v), DTT 10 mM, Bio-lyte pH 3-10 0.2%(w/v), bromophenol blue 0.001%(w/v)) and applied to a 17 cm strip (ReadyStrip™ IPG Strips pH 3-10, Bio-Rad Laboratories Inc., USA). The strip was hydrated for 12 hours at 20° C. and isoelectric focusing was performed. Then, the strip was dipped in equilibrated buffer I (urea 6M, SDS 2%(w/v), Tris—HCl(pH 8.8) 0.375M, glycerol 20%(v/v), DTT 130 mM) for about 15 minutes while shaking, and then was dipped in equilibrated buffer II (urea 6M, SDS 2%(w/v), Tris—HCl(pH 8.8) 0.375M, glycerol 20%(v/v), iodoacetarnide 135 mM, bromophenol blue 3.5M) for about 15 minutes while shaking. The strip was loaded on SDS gel to carry out separation by molecular weight.

The two-dimensional gel was stained with a silver staining kit (Amersham Biosciences, Uppsala, Sweden), scanned with a scanner (GS710 Calibrated Imaging Densitometer, Bio-Rad Laboratories Inc., USA) and subjected to a quantitative analysis of protein by Melanie II software (Bio-Rad Laboratories Inc., USA). Also, for protein identification, desired proteins were taken selectively from the two-dimensional gel, washed, dried in vacuo and reacted with trypsin for 8 hours or more at 37° C. Then, peptides cut by trypsin were measured for their molecular weights using MALDI-TOF MS(Matrix Assisted Laser Desorption/Ionization Time of Flight mass spectrometer) (Voyager™ Biospectrometry, Perseptive Biosystems Inc., USA). The protein levels before leptin expression were compared with those of the maximum content of leptin.

As a result, it was shown that synthesis of substantially all of the amino acids was inhibited after leptin gene expression. Particularly, the levels of enzymes involved in the synthesis of serine family amino acids (CysK, GlyA) were considerably reduced by excessive production of leptin protein, in which GlyA was reduced by 2.5 times and CysK was reduced by 2.3 times (FIG. 1). From these, it was noted that the biosynthesis of serine family amino acids was considerably impeded by the excessive production of leptin and thus, in order to promote metabolism related to reduced biosynthesis of serine family amino acids in the strain producing leptin, the serine-rich protein, the cysK gene encoding the CysK protein which is a critical enzyme in this pathway was to be introduced.

### EXAMPLE 2

Preparation of Recombinant Plasmid with cysK Gene Introduced

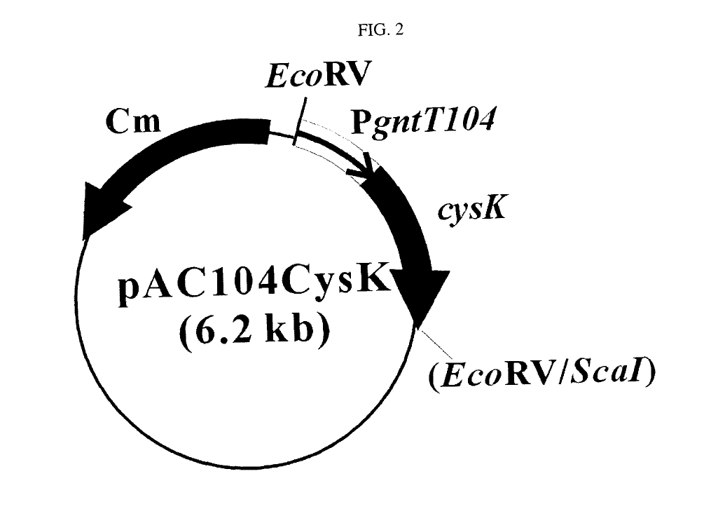

The recombinant plasmid pAC104CysK to express the CysK protein was prepared as follows: Firstly, polymerase chain reaction (PCR) was conducted using the E. coli BL21(DE3) chromosome as a template, and primer 1: 5′-gcgaattcatgagtaagatttttgaagataa-3′(SEQ ID NO: 1) and primer 2: 5′-gcgaattctatatactgttgcaattctttctc-3′(SEQ ID NO: 2). Here, the first denaturation was conducted once at 95° C. for 5 minutes and the second denaturation was conducted by repeating 30 cycles of holding at 95° C. for 50 seconds, annealing at 55° C. for 1 minute and extension at 72° C. for 1 minute and 30 seconds, and then the final extension was once conducted at 72° C. for 5 minutes. The cysK gene thus obtained was cut with the restriction enzyme EcoRI and the resulting segment was inserted into the plasmid p10499A (Park et al., FEMS Microbiol. Lett., 214:217-22, 2002) having the gntT104 promoter (Peekhaus and Conway, J. Bacteriol., 180:1777-85, 1998), which had been digested with the same restriction enzyme, to form the plasmid p104CysK. Then, the plasmid was cut with the restriction enzymes EcoRV and ScaI and cloned into the plasmid pACYC 184 digested with the restriction enzyme EcoRV. The product was transformed into E. coli XL1-blue to prepare recombinant plasmid pAC104CysK(FIG. 2).

### EXAMPLE 3

Preparation of Recombinant Plasmid with IL-12p40 Gene Introduced

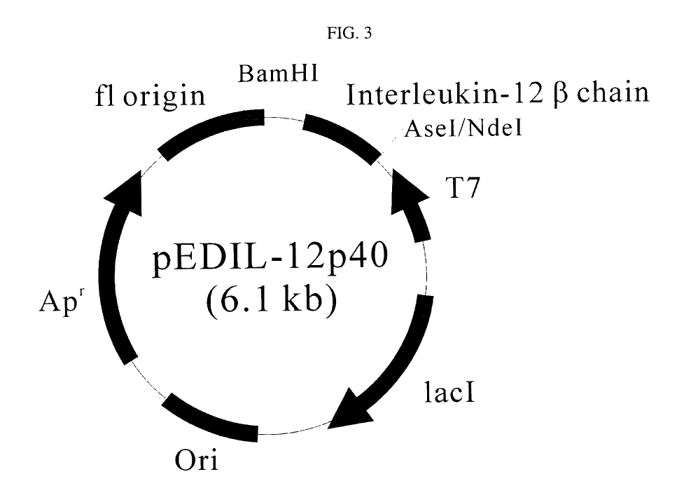

In order to express IL-12p40(interleukin 12 β chain) protein, the recombinant plasmid pEDIL-12p40 was prepared as follows. PCR was conducted using plasmid pUC 18/p40 including human interleukin β chain gene as a template, and primer 3: 5′-ggctagcattaatgatatgggaactgaagaaagat-3′(SEQ ID NO: 3) and primer 4: 5′-gccggatccttattaactgcagggcacaga-3′ (SEQ ID NO: 4) by following the same procedures as in Example 2 to obtain the IL-12p40 gene. The gene was digested with restriction enzymes AdeI and BamHI. The resulting segment was inserted into the leptin expression vector (Jeong and Lee, Appl. Environ. Microbiol., 65:3027-32, 1999), which had been digested with restriction enzymes NdeI and BamHI, to form plasmid pEDIL-12p40 (FIG. 3).

### EXAMPLE 4

Production of Human Leptin Protein by Coexpression System of cysK

The recombinant plasmid pAC104CysK prepared in Example 2 and the conventional leptin expression plasmid pEDOb5 (Jeong and Lee, Appl. Environ. Microbiol. 65, 3027-32, 1999) were transformed simultaneously into E. coli BL21(DE3) to prepare E. coli BL21(DE3)(pEDOb5)(pAC104CysK). This recombinant E. coli was cultured to produce leptin protein. Here, E. coli BL21(DE3)(pEDOb5) transformed with only the conventional leptin expression plasmid pEDOb5 as a control was cultured under the same conditions to produce leptin protein.

Each of the transformed E. coli strains was inoculated into a 10 mL of R/2 medium (KH₂PO₄ 6.75 g/L, (NH₄)₂HPO₄ 2 g/L, citric acid 0.85 g/L, trace metal solution (HCl 5M, FeSO₄•7H₂O 10 g/L, CaCl₂ 2 g/L, ZnSO₄•7H₂O 2.2 g/L, MnSO₄•5H₂O 0.54 g/L, CuSO₄•5H₂O 1 g/L, (NH₄)Mo₇O₂₄•4H₂O 0.1 g/L, Na₂B₄O₇•10H₂O 0.02 g/L), 5 mL/L, MgSO₄•7H₂O 0.7 g/L) with 10 g/L of glucose, cultured at 37° C. for overnight, transported to 200 mL of R/2 medium with 10 g/L of glucose and cultured at 37° C. for 8 hours. Then, 200 mL of the recombinant E. coli which had been cultured in the R/2 medium was inoculated into 1.8L of R/2 medium with 10 g/L of glucose and cultured in an incubator kept at 37° C. and pH 6.88 while supplying a stock solution containing 700 g/L of glucose and 20 g/L of MgSO₄•7H₂O. Here, the stock solution was supplied according to changes of pH. For instance, when pH of the medium was 6.88 or more, the stock solution was automatically adjusted and supplied at rate of 10 mL/min so that the glucose concentration in the fermentation chamber would be 0.7 g/L. Air and pure oxygen were automatically adjusted and supplied to maintain the dissolved oxygen (DO) in the medium at 40%. When the optical density (O.D.) of the culture broth as measured at 600 nm using a spectrophotometer was 30, 1 mM of IPTG(isopropyl-β-thiogalactoside) was added thereto to induce expression of leptin protein. In all the cultures, 100 mg/L of ampicillin and 30 mg/IL of chloramphenicol to stabilize plasmids.

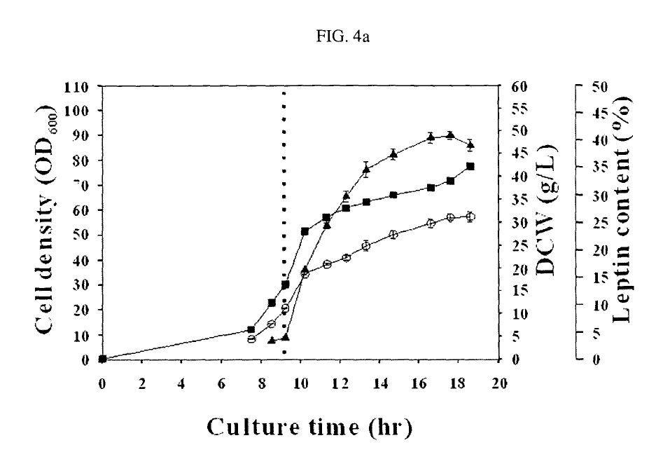

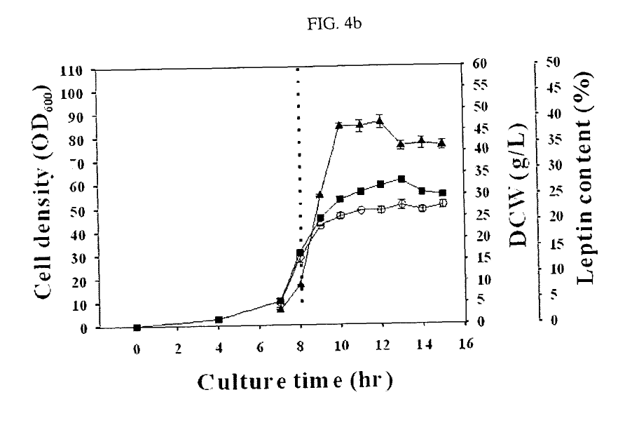

After inducing the expression of leptin protein, the culture broth was taken at every hour. Each aliquot was regularly diluted to be optical density (O.D.) of 5 and centrifuged at 6000 rpm for 5 minutes at 4° C. to form precipitates. The precipitates were suspended in 200 μl of TE buffer (Tris—HCl 10 mM, EDTA 1 mM) and subjected to 12% SDS-PAGE analysis according to a known method (FIG. 4a and FIG. 4b). FIG. 4a is a graph showing changes in cell density, dry cell weight and foreign protein quantity according to the culture time, when the control group is cultured and FIG. 4b is a graph showing changes in cell density, dry cell weight and foreign protein quantity according to the culture time, when the recombinant E. coli

BL21(DE3)(pEDOb5)(pAC104CysK) that can produce leptin is cultured, in which (▪) represents the optical density of cells, (∘) represents the dry cell weight and (▴) represents the amount of prepared leptin. As shown in FIG. 4a, when the leptin expression plasmid was expressed alone, the expression of leptin reached the maximum after 8 hours from induction. From this, it was found that the production yield reached 0.457 g/L•h. On the other hand, as shown in FIG. 4b, when the leptin expression plasmid was coexpressed along with the pAC104CysK, the expression of leptin reached the maximum after 2 hours from induction. From this, it was found that the production yield reached 1.56 g/L•h.

Consequently, it was proved that the method for producing serine-rich protein by coexpression of the cysK gene according to the present invention could increase the leptin productivity by about 3.4 times as compared to the conventional method.

### EXAMPLE 5

Production of Serine-Rich Protein by cysK Coexpression System

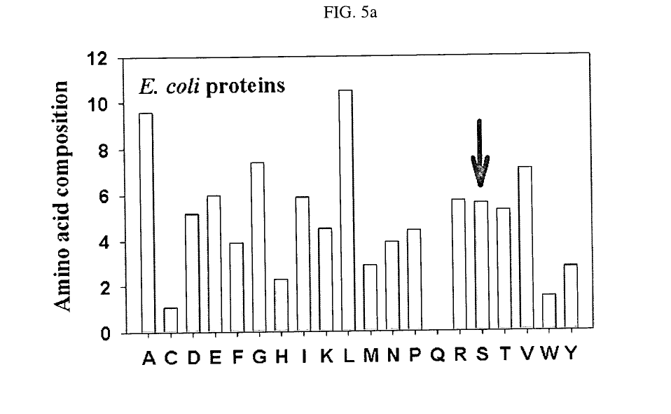

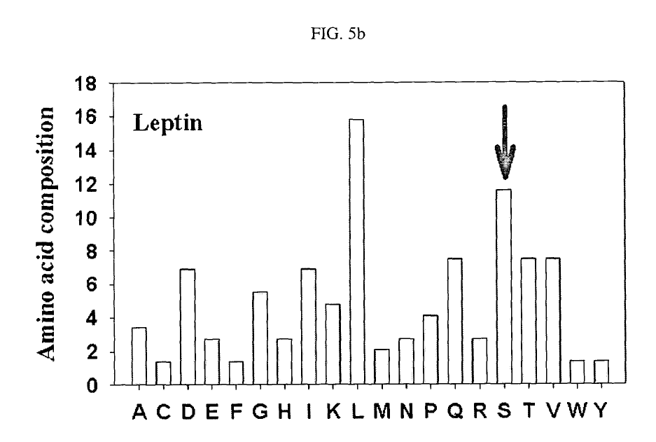

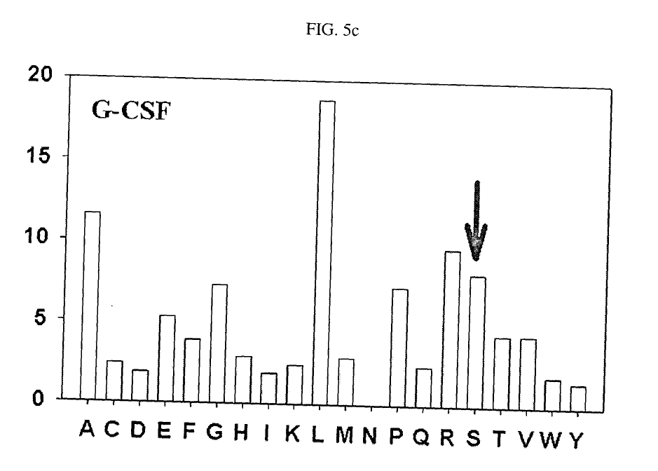

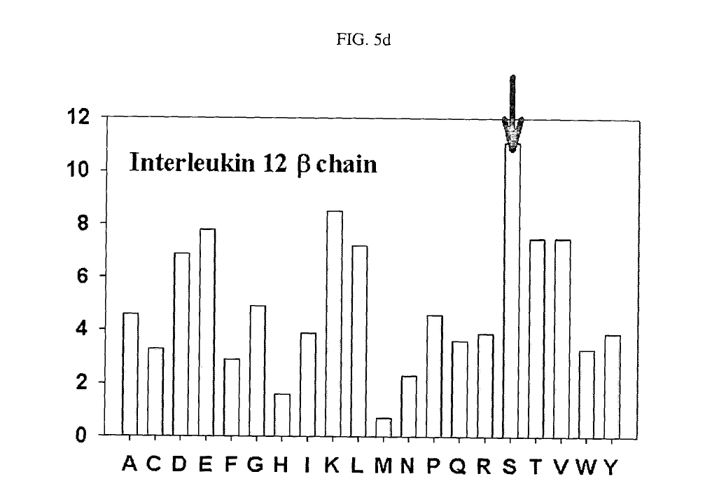

According to reports, leucine and alanine are prevalent in the average composition of amino acids of E. coli proteins (leucine 10.5% and alanine 9.6%) and serine is 5.6% on the average (FIG. 5a, FIG. 5b, FIG. 5c and FIG. 5d). FIGS. 5a to 5d are graphs showing compositional ratio of amino acids of proteins known up to date, in which FIG. 5a shows compositional ratio of amino acids of E. coli proteins, FIG. 5b shows compositional ratio of amino acids of leptin, FIG. 5c shows compositional ratio of amino acids of G-CSF and FIG. 5d shows compositional ratio of amino acids of IL-12p40.

As shown in FIG. 5b, the leptin protein, which is one of typical serine-rich proteins, comprises exceptionally much serine amino acid, in which the compositional ratio of serine is 11.6%. As shown in FIG. 5c, another known protein hG-CSF (human granulocyte-colony stimulating factor) comprises mainly 19% of leucine and 12% of alanine which are similar to proteins in E. coli, though it contains 8.2% of serine. As shown in FIG. 5d, another protein IL-12p40 which is also known as a serine-rich protein contains 11.1% of serine.

In order to confirm if the results of Example 4 can be applied to production of all the serine-rich proteins, the present inventors subjected hG-CSF and the serine-rich protein IL-12p40 to the same method to produce proteins (Jeong and Lee, Protein Expr. Purif., 23:311-8, 2001).

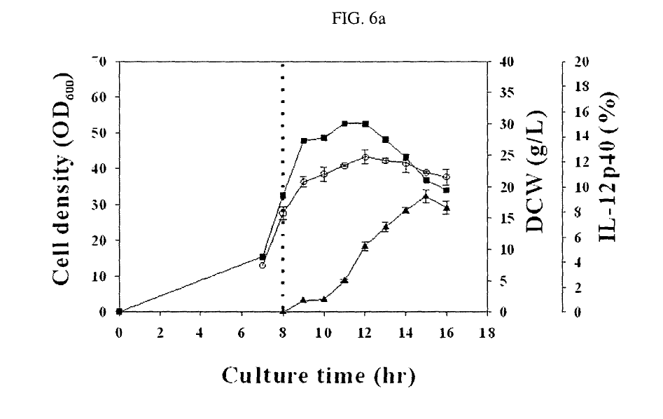

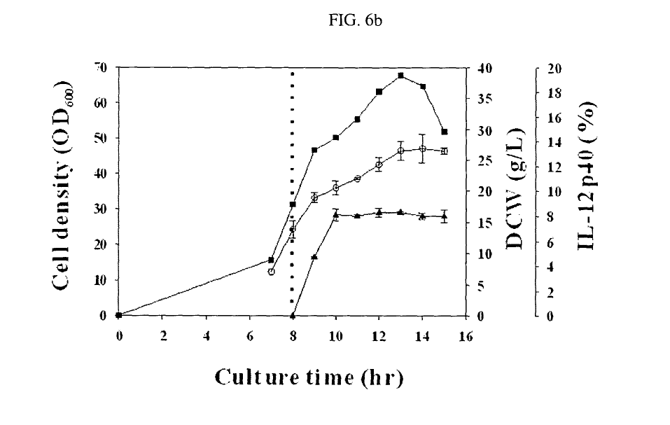

For the hG-CSF, the result was not similar to that of Example 4 because the protein production reached to the peak in a short period of time (3 hours) without co-expression of the cysK gene. However, IL-12p40 showed increasing effects of productivity similar to leptin by coexpression of cysK gene (FIG. 6a and FIG. 6b).

FIG. 6a is a graph showing changes in cell density, dry cell weight and foreign protein quantity according to the culture time, when the IL-12p40-producing recombinant E. coli, BL21(DE3)(pEDIL-12p40), is cultured and FIG. 6b is a graph showing changes in cell density, dry cell weight and foreign protein quantity according to the culture time, when the recombinant E. coli BL2 1 (DE3)(pEDIL-12p40)(pAC104CysK) that can produce IL-12p40 and coexpress the cysK gene, is cultured, in which (▪) represents the optical density of cells, (∘) represents the dry cell weight and (▴) represents the amount of prepared interleukin 12 β chain. As shown in FIG. 6a, when IL-12p40 was produced according to the method of Example 4 except for using pEDIL-12p40 prepared in Example 3, the expression of IL-12p40 reached the peak after 7 hours from induction. From this, it was found that the production yield was 0.090 g/L•h. On the other hand, as shown in FIG. 6b, when IL-12p40 was produced according to Example 4 except for using pAC104CysK prepared in Example 2 and pEDIL-12p40 prepared in Example 3, the expression reaches the peak after 2 hours from induction, at the maximum yield of 0.349 g/L•h.

Consequently, it was proved that the method for producing serine-rich protein by coexpression of the cysK gene according to the present invention could increase the IL-12p40 productivity by about 3.9 times as compared to the conventional method.

As described above, a particular part of the present invention is explained in detail. However, it is apparent to those skilled in the art that such concrete description is only for preferred embodiments and the present invention is not limited thereto. For example, as a method for overexpressing cysteine synthase, introduction of the cysK gene into a foreign protein expression vector or fusion into a chromosome of a host cell may achieve the same effect if the expression amount of the cysK gene. Therefore, the actual scope of the present invention is defined by the attached claims and equivalents thereof.

Industrial Applicability

As described in detail and proven, the present invention provides a method for preparing a foreign protein comprising culturing a bacterium containing the cysK gene and a gene encoding the foreign protein. More particularly, the present invention provides a method for preparing a serine-rich protein comprising culturing a bacterium transformed with an expression vector containing a gene of a serine-rich foreign protein and an expression vector containing the cysK gene, or a bacterium transformed with an expression vector containing both the cysK gene and a gene encoding a serine-rich foreign protein and isolating the foreign protein therefrom.

According to the present invention, it is possible to considerably reduce the time required to reach the maximum protein production when a serine-rich foreign protein is produced using a recombinant E. coli. Therefore, the present invention is expected to be widely used to increase the production yield of a serine-rich foreign protein.

What is claimed is:

1. A process for preparing a foreign protein comprising the steps of culturing a bacterium containing a cystein synthase (cysK) gene and a gene encoding the foreign protein in a culture medium thereby producing the foreign protein; and harvesting the foreign protein.

2. The process according to claim 1, wherein the bacterium is one which has been transformed with a vector containing both the cysK gene and the gene encoding the foreign protein.

3. The process according to claim 1, wherein the bacterium is one which has been transformed with a vector containing the cysK gene and a vector containing the gene encoding a foreign protein.

4. The process according to claim 1, wherein the cysK gene is derived from E. coli.

5. The process according to claim 1, wherein the foreign protein is a serine-rich protein.

6. The process according to claim 5, wherein the serine-rich protein is leptin or IL-12p40(interleukin 12 β chain).

7. A recombinant vector comprising both a cysK gene and a gene encoding a foreign protein.

8. A bacterium transformed with a recombinant vector according to claim 7.

9. A bacterium transformed with a vector containing a cysK gene and a vector containing a gene encoding a foreign protein.

10. The recombinant vector according to claim 7, which is selected from plasmid pAC104CysK as shown in FIG. 2, or plasmidpEDIL-12p40 as shown in FIG. 3.

11. The process according to claim 2, where in the cysK gene is derived from E. coli.

12. The process according to claim 3, where in the cysK gene is derived from E. coli.

13. The process according to claim 2, wherein the foreign protein is a serine-rich protein.

14. The process according to claim 3, wherein the foreign protein is a serine-rich protein.
